(19) United States
(12) Plant Patent Application Publication
(10) Pub. No.: US 2002/0194666 P1
(43) Pub. Date: Dec. 19, 2002

(54) 'LITTLE PHILLIP NO. 1' BERMUDAGRASS

(76) Inventors: Larry Herrington, Florien, LA (US); Clyde Sneed, Many, LA (US)

Correspondence Address: PATENT DEPARTMENT, TAYLOR, PORTER, BROOKS & PHILLIPS, L.L.P, P.O. BOX 2471, BATON ROUGE, LA, 70821-2471, US

(21) Appl. No.: 09/855,065

(22) Filed: May 14, 2001

(51) Int. Cl. A01H 5/00

(52) U.S. Cl. PLT/389

(57) ABSTRACT

An improved bermudagrass plant named “Little Phillip No. 1” is disclosed having superior properties. Phillip is characterized by its moderate stem size, reduced rate of leaf senescence, and superior forage production.

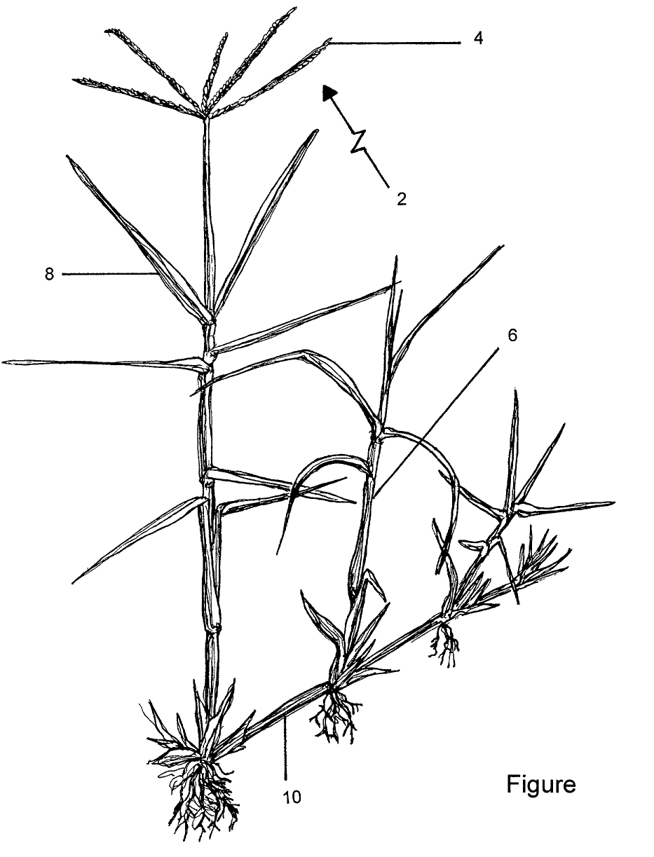

This invention pertains to a new and distinct variety of hybrid bermudagrass which has superior forage production, spring growth, and early stolon development.

This new and distinct variety is identified as “Little Phillip No. 1” (“Phillip”). Phillip is a naturally-occurring, vegetatively propagated variety of a hybrid bermudagrass, Cynodon dactylon (L.) Pers. Phillip demonstrates superior forage production, spring growth, and early stolon development over other available hybrid bermudagrass varieties. Phillip is characterized by its moderate stem size, reduced rate of leaf senescence, and superior forage production.

### BRIEF DESCRIPTION OF THE DRAWING

The FIGURE depicts the growth form of the new and distinct variety of bermudagrass identified as “Little Phillip No. 1.”

Phillip was discovered in a cultivated hay field of Alicia bermudagrass (“Alicia”) on a farm owned by Clyde Sneed, located near Many, La. (911 address: 3271 Corleyville Road, Many, La. 71449) (West Louisiana Coastal Plain area). Phillip was initially observed by Clyde Sneed in a 10 acre field of Alicia as two small inclusions occupying an area of about 4-5 ft². During the next five years Phillip vigorously spread into areas previously occupied by Alicia. Phillip now occupies about 2 acres of the 10 acre field, since it has out-competed Alicia. The novel bermudagrass is suitable for use in farming areas for hay fields, erosion control, and pastures.

In September 1996, Phillip was vegetatively propagated for experimental purposes. Stolon samples were transferred by Larry Herrington to two small plots (approximately ¼ of an acre each) at two separate locations: Clyde Sneed's farm and a neighboring farm owned by Larry Herrington. These sites were plowed and weed-free before transfer of the vegetative planting material. Approximately one month after planting the samples, the sites were mowed to control weed competition. In the winter of 1996-1997, broiler litter (about 1 ton per acre) was applied to the sites as a fertilizer. Both sites were sprayed with the herbicide 2, 4-D (O'Neal's Feeders Supply, Inc., DeRidder, La.) around April 1997 to control broad-leaf weeds. During that period, the vigorous establishment and impressive growth of Phillip was particularly apparent.

It is believed that Phillip most likely originally developed from seed, and that Alicia is probably at least one of the parents of Phillip. Phillip is presumed to be either a natural hybrid between Alicia and another bermudagrass (common bermudagrass is widely distributed in the area) or the outcome of a self-fertilized seed of Alicia (which rarely produces fertile seed). (Phillip could be the result of a mutation, or of a natural recombination of Alicia genes.)

The FIGURE depicts the growth form of the above ground vegetative growth of Little Phillip No. 1. Generally, Phillip is similar in appearance to other robust hybrid bermudagrass varieties. It forms a moderately dense sod with vegetative growth attaining a height of about 1 ft. Seedheads (inflorescences) 2 may extend up to about 18 inches in height. These seedheads 2 can have from five to seven branches 4; however, inflorescences 2 having five branches 4 typically occur most frequently. The branches 4 are about 6-7 cm long. Vegetative stems 6 are typically flattened and range from 2-4 mm wide. Leaf blades 8 are generally from 3-6 mm wide with most leaf lengths ranging from 50-100 mm. Stolons 10 typically reach more than 1 m (several feet) in length.

Tests Conducted

To confirm that Phillip was a new and distinct variety of bermudagrass, in October of 1996 about 10 to 12 stolon samples from Phillip, having lengths between 18 and 24 inches, were submitted to the Louisiana Agricultural Experiment Station (Rosepine Research Station), located in Rosepine, La., for evaluation and comparison with leading bermudagrass varieties. Three vegetatively propagated bermudagrass varieties, Russell, Tifton-85 and Jiggs were selected to conduct comparison tests with Phillip. These three varieties were chosen because they represent superior commercially available bermudagrass varieties in the West Louisiana Coastal Plain area, and naturally provide a range in growth type among varieties.

Established stands of each grass were evaluated in field plot clipping trials for forage production and stand survival. Each grass entry was planted in individual plots having dimensions of 7 ft×20 ft, using a Bowie fine sandy loam soil. The plots were placed 5 ft apart. At the beginning of each growing season (1998 to 2000), each plot was fertilized with 50 pounds per acre of nitrogen, P₂O₅, and K₂O (about 300 pounds per acre of 16% N, 16% P₂O₅, and 16% K₂O (16-16-16 mixed fertilizer)). After each forage harvest, with the exception of the last harvest of each year, 50 pounds per acre of nitrogen was again applied to the soil in each plot. Harvests were made throughout the 1998 to 2000 growing seasons. Tests were conducted on forage yield samples to determine the dry matter percentage. Also, crude protein and in vitro digestibility analyses were conducted by the Forage Quality Laboratory at the Louisiana Agricultural Experiment Station (Southeast Research Station), located in Franklinton, La.

Forage dry matter yields of the hybrid bermudagrass varieties for the 1998 growing season are shown in Table 1.

TABLE 1

| | Harvest date | | | | |
|---|---|---|---|---|---|
| Variety | May 11 | June 18 | July 21 | Aug. 28 | Oct. 21 |
| | pounds per acre | | | | |
| Russell | 870 | 1010 | 630 | 1590 | 2570 |
| Jiggs | 730 | 1160 | 530 | 2620 | 3290 |
| Tifton-85 | 480 | 1400 | 770 | 2710 | 2610 |
| Phillip | 1410 | 1070 | 680 | 3680 | 3000 |

Forage dry matter yields of the hybrid bermudagrass varieties for the 1999 growing season are shown in Table 2. Forage production was more uniformly distributed throughout this growing season than the 1998 growing season, probably due primarily to more uniform rainfall through the growing season.

TABLE 2

| | Harvest date | | | | |
|---|---|---|---|---|---|
| Variety | May 19 | June 16 | July 16 | Aug. 30 | Nov. 3 |
| | pounds per acre | | | | |
| Russell | 1020 | 1690 | 1650 | 1210 | 770 |
| Jiggs | 1260 | 2420 | 2760 | 2520 | 2180 |
| Tifton-85 | 1160 | 2570 | 2810 | 2710 | 2230 |
| Phillip | 2860 | 2950 | 3530 | 4360 | 3240 |

Forage dry matter yields of the hybrid bermudagrass varieties for the 2000 growing season are shown in Table 3. Yields were less uniform in 2000 than previous years, probably due primarily to low soil moisture.

TABLE 3

| | Harvest date | | | | |
|---|---|---|---|---|---|
| Variety | May 3 | June 5 | July 13 | Sept. 15 | Oct. 26 |
| | pounds per acre | | | | |
| Russell | 1470 | 1990 | 2100 | 820 | 660 |
| Jiggs | 2090 | 2930 | 4040 | 1580 | 1260 |
| Tifton-85 | 1680 | 2920 | 3450 | 1670 | 840 |
| Phillip | 3210 | 3990 | 3720 | 2920 | 1970 |

Three-year average dry matter yields of the hybrid bermudagrass varieties for the 1998, 1999 and 2000 growing seasons are shown in Table 4. Total forage production for Tifton-85 and Jiggs was over 9,000 pounds per acre; however, Phillip's forage yield was at least 30% more than Tifton-85 and Jiggs, and 53% more than Russell during the three-year period.

TABLE 4

| | Forage yield |
|---|---|
| Variety | pounds per acre per year |
| Russell | 6250 a^{†} |
| Jiggs | 9800 b |
| Tifton-85 | 9350 b |
| Phillip | 13310 c |

^{†}Average yields of varieties followed by a common letter do not differ (P > 0.05) according to Duncan's Multiple Range Test.

Crude protein and in vitro digestibility of the grass varieties for the 1998 and 1999 growing seasons are shown in Table 5. Crude protein did not differ significantly among the varieties. In vitro digestibility, however, was higher in 1998 than in 1999. (Such differences are typical and reflect variations in growing conditions.)

TABLE 5

| | Crude | In vitro digestibility | |
|---|---|---|---|
| Variety | protein^{†} | 1998 | 1999 |
| | % | | |
| Russell | 13.8 | 60.2 c^{‡} | 57.6 a |
| Jiggs | 13.8 | 62.6 b | 57.8 a |
| Tifton-85 | 14.0 | 64.5 a | 57.6 a |
| Phillip | 14.1 | 62.8 b | 57.9 a |

^{†}Varieties did not differ significantly (P > 0.05) in crude protein concentration in either year.
^{‡}Responses among varieties in in vitro digestibility differed between years. Average digestibility values for varieties within each year do not differ (P > 0.05) if followed by a common letter.

Production of Tifton-85 at the first harvest was comparatively low because of its open stand which naturally occurs early in each growing season. (Tifton-85 naturally produces a sparse or open stand with bare soil available for competing plants early in the growing season and after each harvest.)

Distinguishing Morphological Features of Phillip

To assess some of the morphological features of Phillip, measurements of leaf blade length and width were taken of the uppermost fully expanded leaf of the vegetative tillers of all four bermudagrass varieties, as shown in Table 6. The measurements were taken on May 8, 2001, five days after a forage harvest. Five tillers in each of four replicates of each of the bermudagrass varieties were measured. Leaf measurements were subjected to statistical analysis using analysis of variance with significantly different responses (P<0.05) assessed by least significant difference procedures.

TABLE 6

| Variety | Leaf width (mm) | Leaf length (mm) |
|---|---|---|
| Tifton-85 | 4.7 a^{†} | 75.3 a |
| Phillip | 4.3 ab | 79.7 a |
| Jiggs | 4.1 b | 75.2 a |
| Russell | 3.3 c | 59.3 b |
| Least significant difference | 0.46 | 12.2 |

^{†}Measurements within a column followed by a common letter do not differ (P < 0.05) by the least significant difference procedure.

As shown in Table 6, Phillip and Tifton-85 had similar leaf blade dimensions. However, Phillip can be distinguished from Tifton-85 by its dense, closed sod under comparable growing conditions. Another distinguishing feature is that Phillip has a larger number of fully-expanded leaves per unit of area than Tifton-85. Additionally, Tifton-85 stands are more vulnerable to invasion by other plants (e.g., common bermudagrass), than Phillip. Tifton-85 stands tend to grow upright with a substantial amount of bare ground exposed following defoliation. However, Phillip develops stands with more closely spaced plants that are not as readily invaded by other plants, especially during the period immediately following harvest of the forage growth.

Phillip and Jiggs also had similar leaf blade dimensions, as shown in Table 6.

Phillip had longer and wider leaf blades than Russell, as shown in Table 6. Phillip can also be distinguished from Russell by its larger stem size.

Under comparable growing conditions, Phillip was found to exhibit greater rates of vegetative growth and stolon development than Russell, Jiggs and Tifton-85. Additionally, Phillip exhibited a faster rate of plant morphological development than Russell, Jiggs, and Tifton-85.

Phillip Distinguished From Alicia

Under comparable growing conditions, Phillip can be distinguished from Alicia by its larger stem size and reduced rate of leaf senescence in the lower canopy layer of stands. The senescence of the lower leaves in Alicia causes deterioration of forage quality after about four weeks or more of growth. Since the rate of senescence in Phillip is substantially less than the rate of senescence in Alicia, forage of a higher quality is produced by Phillip throughout the canopy for forage of 6 or more weeks of age, depending on growing conditions.

Several additional experiments will be conducted by the Louisiana Agricultural Experiment Station, including evaluations of forage yield and quality in response to the length of growing periods between harvests and response to nitrogen fertilizer rate. Also, comparisons of forage yield and quality with currently used varieties will be made at various locations in Louisiana.

We claim:

1. A bermudagrass plant substantially as described and illustrated in the specification herein.
